(19) United States
(12) Patent Application Publication
(10) Pub. No.: US 2025/0228774 A1
(43) Pub. Date: Jul. 17, 2025

(54) SYSTEMS AND METHODS FOR ACCELERATED HYDROLYSIS OF POLYSACCHARIDE-BASED HYDROGELS

(71) Applicant: Boston Scientific Scimed, Inc., Maple Grove, MN (US)

(72) Inventors: Yen-Hao Hsu, Shrewsbury, MA (US); Heather Cirka, Hudson, MA (US); Marc Plourde, Worcester, MA (US); Cristian Parisi, Boston, MA (US)

(73) Assignee: Boston Scientific Scimed, Inc., Maple Grove, MN (US)

(21) Appl. No.: 19/015,112

(22) Filed: Jan. 9, 2025

Related U.S. Application Data
(60) Provisional application No. 63/620,056, filed on Jan. 11, 2024.

(51) Int. Cl. A61K 9/06; A61K 9/00; A61K 47/12; A61K 47/61

(52) U.S. Cl. CPC A61K 9/06; A61K 9/0019; A61K 47/12; A61K 47/61

(57) ABSTRACT

In some aspects, the present disclosure provides kits for delivering and hydrolyzing crosslinked polysaccharide hydrogels, the kits comprising (a) a hydrogel delivery system configured to deliver a crosslinked polysaccharide hydrogel to a subject, the crosslinked polysaccharide hydrogel comprising polysaccharide molecules that are crosslinked by crosslinks that comprise hydrolysable ester bonds and (b) a hydrogel hydrolysis system configured to deliver a hydrolysis-accelerating catalytic composition to the subject, the hydrolysis-accelerating catalytic composition adapted to accelerate hydrolysis of the hydrolysable ester bonds in the crosslinked polysaccharide hydrogel. In other aspects, the present disclosure provides methods of using the same.

### CROSS-REFERENCE TO RELATED APPLICATIONS

The application claims the benefit of U.S. Provisional Patent Application Ser. No. 63/620,056 filed on Jan. 11, 2024, the disclosure of which is incorporated herein by reference.

### FIELD

The present disclosure relates to systems and methods for accelerated hydrolysis of polysaccharide-based hydrogels. Such systems and methods are useful, for example, in removal of hydrogels from patients.

### BACKGROUND

Injectable hydrogels are a newly emerging class of materials having a variety of medical uses. As one specific example, injectable hydrogels have been used to create or maintain space between tissues in order to reduce side effects of off-target radiation therapy. The hydrogel creates a space between the rectum and the prostate, moving the rectum away from the treatment region. This can help to reduce radiation exposure to the rectum and/or provide other desirable benefits.

An example of a hydrogel-based perirectal spacer material is a hyaluronic acid injectable hydrogel, commercially available as Barrigel®. Barrigel® is cross-linked with 1,4-butanediol diglycidyl ether (BDDE) under alkaline conditions, thereby creating ether bonds between the hyaluronic acid chains, resulting in a three-dimensional network. However, this approach generates covalent bonds without tunable degradation capability.

The use of a hydrogel-based perirectal spacer material in conjunction with prostate radiation therapy is illustrated schematically in FIGS. 1A and 1B. FIG. 1A illustrates a cross-section of the human male anatomy including the prostate 110 and rectal wall 112. When the prostate is treated using radiation therapy there is a higher dose region 114 adjacent to the prostate, which is subjected to high doses of radiation, becoming a lower dose region 116 as one proceeds further from the prostate 110. As illustrated in FIG. 1B, a spacing material 118 can be injected between the prostate 110 and the rectal wall 112, which can push the rectal wall from a higher dose region 114 to a lower dose region 116, thereby reducing injury to the rectal wall.

It can be appreciated by those skilled in the art that placing a spacer into a patient may lead to complications such as patient discomfort, infiltration of the hydrogel into the rectal wall, and urinary retention. Furthermore, misplacement of a spacer may reduce the effectiveness of the spacer. Even though these complications are extremely rare, there is still clinical interest in potentially reversing the placement when needed. Disclosed herein are systems and methods for improving patient comfort, improving the effectiveness of the spacer, addressing spacer misplacement, combinations thereof, and/or other benefits. Such systems may be used, for example, to accelerate the hydrolysis of hydrogels, including acceleration of the hydrolysis of hydrogel-based spacers.

### SUMMARY

In some aspects, the present disclosure provides kits for delivering and hydrolyzing crosslinked polysaccharide hydrogels, the kits comprising (a) a hydrogel delivery system configured to deliver a crosslinked polysaccharide hydrogel to a subject, the crosslinked polysaccharide hydrogel comprising polysaccharide molecules that are crosslinked by crosslinks that comprise hydrolysable ester bonds and (b) a hydrogel hydrolysis system configured to deliver a hydrolysis-accelerating catalytic composition to the subject, the hydrolysis-accelerating catalytic composition adapted to accelerate hydrolysis of the hydrolysable ester bonds in the crosslinked polysaccharide hydrogel.

In some embodiments, the hydrolysis-accelerating catalytic composition comprises a hydrolysis-accelerating catalyst selected from a hydroxide catalyst and an enzymatic protein.

In some embodiments, which can be used in conjunction with the above aspects and embodiments, the hydrolysis-accelerating catalytic composition is prepackaged a hydrolysis syringe system.

In some embodiments, which can be used in conjunction with the above aspects and embodiments, the hydrogel hydrolysis system further comprises a needle, a flexible tube, or both and wherein the hydrolysis syringe system is configured for coupling to the needle, the flexible tube, or both.

In some embodiments, which can be used in conjunction with the above aspects and embodiments, the hydrogel delivery system comprises a preformed crosslinked polysaccharide hydrogel. In some of these embodiments, the preformed crosslinked polysaccharide hydrogel comprises crosslinked polysaccharide hydrogel particles, which may be prepackaged, for example, in a delivery syringe system. In some of these embodiments, the hydrogel delivery system may further comprise a needle, a flexible tube, or both and the delivery syringe system may be configured for coupling to the needle, the flexible tube, or both.

In some embodiments, which can be used in conjunction with the above aspects and embodiments, the hydrogel delivery system comprises a carboxylic-acid-containing polysaccharide, a polyol, and an ester coupling agent. In some of these embodiments, the hydrogel delivery system is configured to form a mixture of the carboxylic-acid-containing polysaccharide, the polyol, and the ester coupling agent and to deliver the mixture to a subject, whereupon the carboxylic-acid-containing polysaccharide and the polyol form the crosslinks with one another. In some of these embodiments, the hydrogel delivery system comprises a delivery syringe system that is configured to form a mixture of the carboxylic-acid-containing polysaccharide, the polyol, and the ester coupling agent and to deliver the mixture to a subject whereupon the carboxylic-acid-containing polysaccharide and the polyol form the crosslinks with one another. For example, the delivery syringe system may comprise a dual barrel syringe device for creating and delivering the mixture of the carboxylic-acid-containing polysaccharide, the polyol, and the ester coupling agent.

In some embodiments, which can be used in conjunction with the above aspects and embodiments, the hydrogel delivery system comprises a hydroxyl-containing polysaccharide, a polycarboxylic acid molecule, and an ester coupling agent. In some of these embodiments, the hydrogel delivery system is configured to form a mixture of the hydroxyl-containing polysaccharide, the polycarboxylic acid molecule, and the ester coupling agent and to deliver the mixture to a subject whereupon the hydroxyl-containing polysaccharide and the polycarboxylic acid molecule form the crosslinks with one another. In some of these embodiments, the hydrogel delivery system comprises a delivery syringe system that is configured to form a mixture of the hydroxyl-containing polysaccharide, the polycarboxylic acid molecule, and the ester coupling agent and to deliver the mixture to a subject whereupon the hydroxyl-containing polysaccharide and the polycarboxylic acid molecule form the crosslinks with one another. For example, the delivery syringe system may comprise a dual barrel syringe device for creating and delivering the mixture of the hydroxyl-containing polysaccharide, the polycarboxylic acid molecule, and the ester coupling agent.

Other aspects of the present disclosure pertain to methods that comprise (a) delivering a crosslinked polysaccharide hydrogel to a subject, the crosslinked polysaccharide hydrogel comprising polysaccharide molecules that are crosslinked by crosslinks that comprise hydrolysable ester bonds and (b) delivering a hydrolysis-accelerating catalytic composition to the subject such that the hydrolysis-accelerating catalytic composition contacts the crosslinked polysaccharide hydrogel, the hydrolysis-accelerating catalytic composition acting to accelerate hydrolysis of the hydrolysable ester bonds in the crosslinked polysaccharide hydrogel.

In some embodiments, the method comprises delivering the crosslinked polysaccharide hydrogel to the subject via a delivery syringe system coupled to a needle; disconnecting the delivery syringe system from the needle; attaching a hydrolysis syringe system to the needle, the hydrolysis syringe system including the hydrolysis-accelerating catalytic composition disposed in a syringe barrel; and injecting the hydrolysis-accelerating catalytic composition into the subject.

In some embodiments, which can be used in conjunction with the above aspects and embodiments, the method further comprises observing a complication associated with the delivering of the crosslinked polysaccharide hydrogel to the subject prior to injecting the hydrogel hydrolysis material into the subject. For example, the complication may include misplacement of the crosslinked polysaccharide hydrogel and/or subject discomfort.

In some embodiments, which can be used in conjunction with the above aspects and embodiments, the method further comprises delivering a replacement crosslinked polysaccharide hydrogel into the subject.

In some embodiments, which can be used in conjunction with the above aspects and embodiments, the crosslinked polysaccharide hydrogel provides spacing between tissues of the subject and the method further comprises treating the subject with radiation therapy.

The above and other aspects, embodiments, features and benefits of the present disclosure will be readily apparent from the following detailed description.

### BRIEF DESCRIPTION OF THE DRAWINGS

FIGS. 1A and 1B schematically illustrate the use of a hydrogel-based perirectal spacer material in conjunction with prostate radiation therapy, in accordance with the prior art.

FIG. 2A schematically illustrates the formation of a crosslinked polysaccharide having ester-bond-containing crosslinks, in accordance with an embodiment of the present disclosure.

FIG. 2B schematically illustrates the use of an esterase to accelerate the hydrolysis of the crosslinked polysaccharide of FIG. 2A, in accordance with an embodiment of the present disclosure.

FIG. 3 schematically illustrates a method of forming a non-iodinated polymeric polyol, in accordance with an embodiment of the present disclosure.

FIG. 4 schematically illustrates a method of forming an iodinated polymeric polyol, in accordance with an embodiment of the present disclosure.

FIG. 5 schematically illustrates the formation of an iodinated crosslinked polysaccharide having ester-bond-containing crosslinks, in accordance with an embodiment of the present disclosure.

FIG. 6 is a schematic depiction a region of the human body.

FIG. 7 depicts the placement of a crosslinked polysaccharide hydrogel spacer in the region of the human body depicted in FIG. 6.

FIG. 8 depicts the delivery of a hydrolysis-accelerating catalytic composition to the crosslinked polysaccharide hydrogel spacer of FIG. 7.

FIG. 9 depicts the region of the human body of FIG. 8 after accelerated hydrolysis of the crosslinked polysaccharide hydrogel spacer.

FIG. 10 schematically illustrates a pre-loaded syringe that contains an injectable hydrolysis-accelerating catalytic composition, in accordance with an embodiment of the present disclosure.

FIG. 11 schematically illustrates a delivery device for delivering a crosslinked polysaccharide hydrogel, in accordance with an embodiment of the present disclosure.

FIG. 12 schematically illustrates a delivery device for forming a crosslinked polysaccharide in situ, in accordance with an embodiment of the present disclosure.

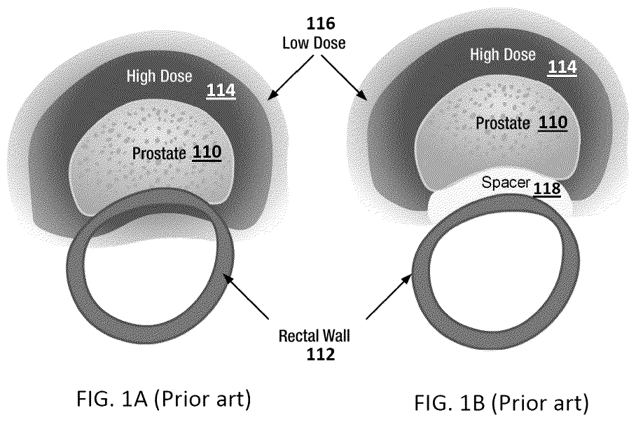

### DETAILED DESCRIPTION

In some aspects, the present disclosure pertains to a method of accelerating the hydrolysis and breakdown of a crosslinked polysaccharide hydrogel in situ (i.e., within a subject). The crosslinked polysaccharide hydrogel comprises crosslinks between polysaccharide chains within the crosslinked polysaccharide hydrogel, which crosslinks contain hydrolysable ester bonds. The method comprises contacting the crosslinked polysaccharide hydrogel with a hydrolysis-accelerating catalytic composition that is adapted to accelerate hydrolysis of the ester bonds within the crosslinks. Contacting may include, for example, applying the hydrolysis-accelerating catalytic composition onto a surface of the crosslinked polysaccharide hydrogel, injecting the hydrolysis-accelerating catalytic composition into the crosslinked polysaccharide hydrogel, and so forth

As used herein, a “hydrogel” is a crosslinked polymer that contains water or can absorb water but does not dissolve when placed in water.

Preferred subjects include mammalian subjects, particularly human subjects.

In some of these embodiments, a crosslinked polysaccharide is provided in which a carboxylic-acid-containing polysaccharide is covalently bonded with a polyol through ester groups. For example, the crosslinked polysaccharide may be formed by an ester-forming reaction between carboxylic acid groups of the carboxylic-acid-containing polysaccharide and hydroxyl groups of the polyol, which acts as a crosslinker for the carboxylic-acid-containing polysaccharide. The ester coupling between the carboxylic acid and hydroxyl groups may be performed in the presence of a suitable coupling agent, for example, a carbodiimide coupling agent, such as N,N′-dicyclohexylcarbodiimide (DCC), 1-ethyl-3-(3-dimethylaminopropyl) carbodiimide (EDC), or N,N′-diisopropylcarbodiimide (DIC), among others. The ester linkage that is formed is hydrolysable.

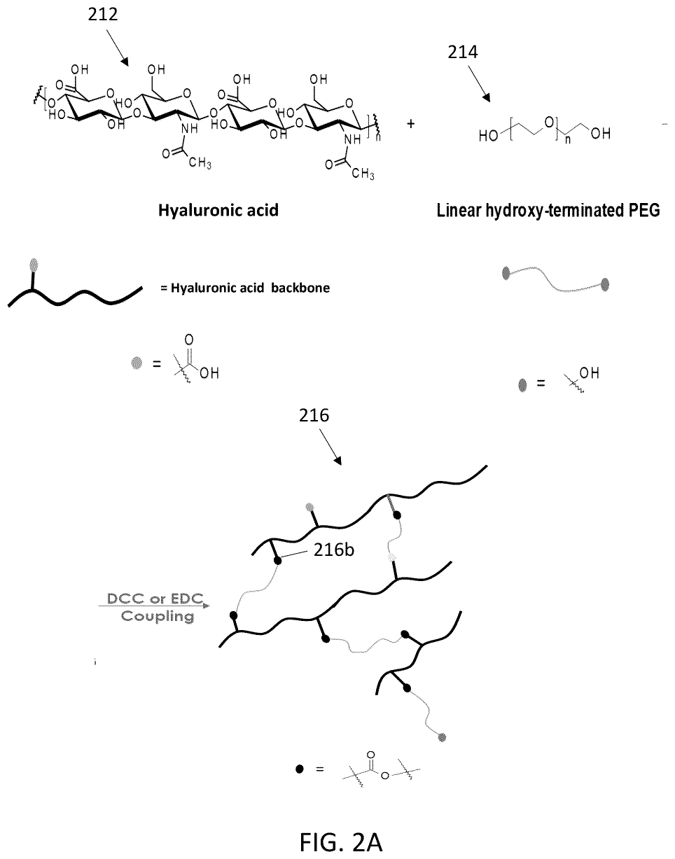

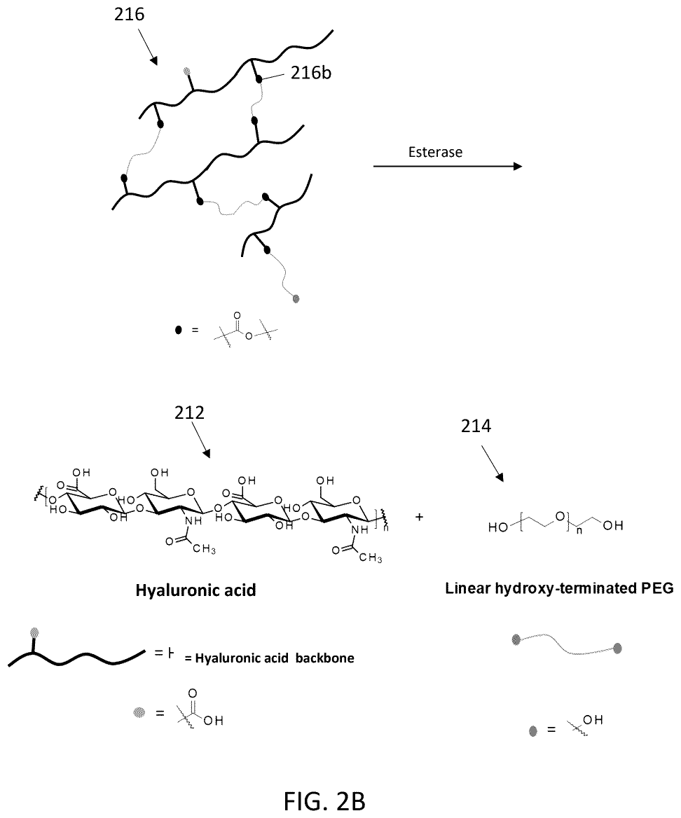

For example, referring now to FIG. 2A, carboxyl groups of a carboxylic-acid-containing, specifically, hyaluronic acid (212) may be reacted with hydroxyl groups of a polyol molecule, specifically a linear bis-hydroxyl-terminated polyethylene glycol (PEG) molecule (214), in an ester coupling reaction in the presence of a suitable coupling agent, such as DCC or EDC, to form a crosslinked polysaccharide (216) in which hyaluronic acid chains are crosslinked to one another through PEG-containing crosslinks that comprise hydrolysable ester bonds (216b). Such a crosslinked polysaccharide hydrogel (216) may be formed in vivo within a subject or may be formed ex vivo and subsequently delivered to a subject.

In the event that it is desirable to remove the crosslinked polysaccharide from the subject (e.g. because the crosslinked polysaccharide is causing discomfort, because the crosslinked polysaccharide is improperly placed, etc.), the crosslinked polysaccharide may be contacted with a hydrolysis-accelerating catalytic composition that is adapted to break down ester bonds in the crosslinks. For example, and with reference to FIG. 2B, a crosslinked polysaccharide hydrogel (216) like that of FIG. 2A, may be contacted with a hydrolysis-accelerating catalyst, specifically, an esterase, which breaks down ester bonds (216b) in the PEG-containing crosslinks, thereby releasing the hyaluronic acid (212) and linear bis-hydroxyl-terminated polyethylene glycol (PEG) (214) from one another. The degradation products shown are non-toxic, biocompatible and bioresorbable. Although 100% of the ester bonds (216b) are shown to be broken in FIG. 2B, substantial breakdown of the hydrogel may be accomplished by breaking less than 100% of the ester bonds (216b). For example, anywhere from 1% to 100%, preferably 10% to 100%, of the ester bonds (216b) may be broken down in some embodiments.

Hydrolysis-accelerating catalytic compositions for use in the present disclosure comprise one or more one hydrolysis-accelerating catalysts. Illustrative hydrolysis-accelerating catalysts include hydroxides such as NaOH and KOH which accelerate ester bond hydrolysis at low concentrations and enzymatic proteins that break down ester bonds, such as esterases. Esterases may be non-specific and be capable of hydrolyzing a variety of ester bonds or esterases may show selective binding to a specific type of ester bond. Enzymatic proteins may be selected, for example, from suitable members of the following: a carboxylesterase, peptidase, amidase, acetylcholinesterase, bile-salt-activated esterase, protein-glutamate methylesterase, cholinesterase, carboxymethylene-butenolidase, crystal protein, cutinase, cAMP-regulated D2 protein, 2-hydroxymuconic semialdehyde hydrolase, gut esterase, esterase B1, liver carboxylesterase, esterase 1, esterase B2, esterase 4, esterase 5, esterase 6, arginine esterase, esterase 5A, esterase 5B, esterase 5C, esterase D, juvenile hormone esterase, esterase P, Pi 6.1 esterase, phosphatidylcholine-sterol acyltransferases, porcine pancreatic lipase, lipase 1, lipase 2, lipase 3, lipase 4, lipase 5, triacylglycerol lipase, lipoprotein lipase, pancreatic lipase, hormone sensitive lipase, lactonizing lipase, mono and diacylglycerol lipase, 6-methylsalicylic acid synthase, phenmedipham hydrolase, poly (3-hydroyalkanoate) depolymerase, 2-hydroxy-6-oxo-2,4-heptadienoate hydrolase, tropinesterase, vitellogenin I, vitellogenin II, vitellogenin III, 2-hydroxymuconic semialdehyde hydrolase, acetyl esterase, protein-glutamate methyl-esterase, S-acyl fatty acid synthase thioesterase, acetyl-hydrolase, erythronolide synthase, gramicidin S biosynthesis GRST protein, triglyceride lipase-cholesterol esterase, Candida Antarctica B lipase, and/or the like.

In addition to one or more hydrolysis-accelerating catalysts, the hydrolysis-accelerating catalytic compositions of the present disclosure may contain additional agents, including therapeutic agents, imaging agents, colorants, tonicity adjusting agents, suspension agents, wetting agents, and pH adjusting agents as detailed below.

Carboxylic-acid-containing polysaccharides for use in the present disclosure include those that contain one or more uronic acid species, such as galacturonic acid, glucuronic acid and/or iduronic acid. Particular examples of carboxylic-acid-containing polysaccharides include alginic acid, hyaluronic acid, pectin, agaropectin, carrageenan, gellan gum, gum arabic, guar gum, xanthan gum, and carboxymethyl cellulose moieties. In some embodiments, the carboxylic-acid-containing polysaccharides may have a number average molecular weight ranging from 1 kDa to 8000 kDa, for example ranging anywhere from 1 kDa to 2.5 kDa to 5 kDa to 10 kDa to 25 kDa to 50 kDa to 100 kDa to 250 kDa to 500 kDa to 1000 kDa to 2000 kDa to 8000 kDa (in other words, ranging between any two of the preceding numerical values).

Polyols for use in accordance with the present disclosure include polyols having two or more hydroxyl groups, for example, containing anywhere from 2 to 100 hydroxyl groups (e.g., having 3 to 4 to 5 to 6 to 7 to 8 to 9 to 10 to 12 to 15 to 20 to 25 to 30 to 40 to 50 to 60 to 70 to 80 to 90 to 100 hydroxyl groups

Polyols for use in the present disclosure include polymeric polyols and non-polymeric polyols.

Polyols for use in the present disclosure include small molecule polyols and large molecule polyols. As used therein, a “small molecule” is one having a molecular weight of less than 2500, in some embodiments less than 1000, in some embodiments less than 500. As used therein, a “large molecule” is one having a molecular weight of 2500 or more.

Polymeric polyols include linear polymers that comprise one or more polymer segments and have a hydroxy-containing moiety linked at each end, a specific example of which is the linear bis-hydroxyl-terminated polyethylene glycol (PEG) of FIG. 2A.

Polymeric polyols also include multi-arm polymers that have a core region and a plurality of polymer arms, each polymer arm comprising one or more polymer segments and each polymer arm having a fixed end linked to the core region and an opposite free end that is linked to a hydroxy-containing moiety.

In certain embodiments, the core region multi-arm polymer comprises a residue of a polyol comprising two or more hydroxyl groups, which is used to form the polymer arms. In certain beneficial embodiments, the core region comprises a residue of a polyol that contains from 2 to 100 hydroxyl groups.

Polymer segments for use in the linear and multi-arm polymers described herein may be selected from any of a variety of synthetic, natural, or hybrid synthetic-natural polymer segments. Examples of polymer segments include those that are formed from one or more monomers selected from the following: C₁-C₆-alkylene oxide monomers (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), and 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments for the polymer arms may be selected, for example, from the following polymer segments: polyether segments including poly(C₁-C₆-alkylene oxide) segments such as poly(ethylene oxide) (PEO) segments (also referred to as polyethylene glycol segments or PEG segments), poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Each polymer segment for use in the polymers of the present disclosure may contain between 2 and 1000 monomer units or more, for example, ranging anywhere from 2 to 5 to 10 to 30 to 70 to 100 to 300 to 700 to 1000 units (in other words, ranging between any two of the preceding values).

In some embodiments of the present disclosure, preformed polymer segments may be attached to a core-forming molecule.

In some embodiments of the present disclosure, a non-iodinated or iodinated polyol such as one or those described below may be used as multi-functional initiator for polymer chain growth, leading to a hydroxy terminated multi-arm polymer. For example, the non-iodinated or iodinated polyol may be used as an initiator for ring-opening polymerization of ethylene oxide to form poly(ethylene oxide) (PEG) segments at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity.

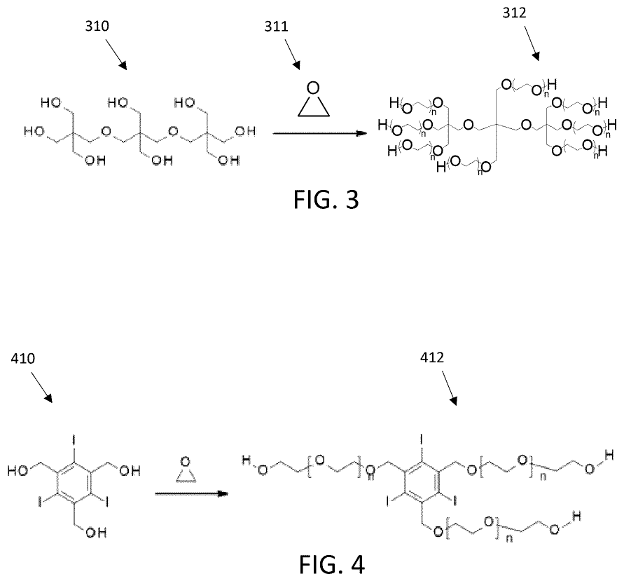

In a particular embodiment shown in FIG. 3, commercially available tripentaerythritol (310) (CAS #78-24-0) can be used as an octa-functional initiator, which undergoes ring-opening polymerization with ethylene oxide (311). The polymerization process leads to poly(ethylene oxide) (PEG) chain growth at each of the eight hydroxyl groups of the tripentaerythritol, forming a hydroxyl-terminated 8-arm-PEG (312) having a tripentaerythritol residue core. In FIG. 3, n is an integer representing the number of monomer units in each polymer segment shown. In other cases, only a single monomer unit is present and n=1.

The strategy shown in FIG. 3 is broadly applicable and can be used in conjunction with a range of polyols, including those described above. In a particular embodiment shown in FIG. 4, an iodinated polyol, 1,3,5-triiodo-2,4,6-tris-hydroxymethylbenzene (410), is used as an initiator, which undergoes ring-opening polymerization with ethylene oxide. The polymerization process leads to poly(ethylene oxide) (PEG) chain growth at each of the three hydroxyl groups of the 1,3,5-triiodo-2,4,6-tris-hydroxymethylbenzene (410). The resulting multi-arm polymer (412) contains three PEG arms that extend from a 1,3,5-triiodo-2,4,6-tris-hydroxymethylbenzene residue core. Each of the PEG arms has a terminal hydroxyl group. In FIG. 4, n is an integer representing the number of monomer units in each polymer segment shown. In other cases, only a single monomer unit is present and n=1.

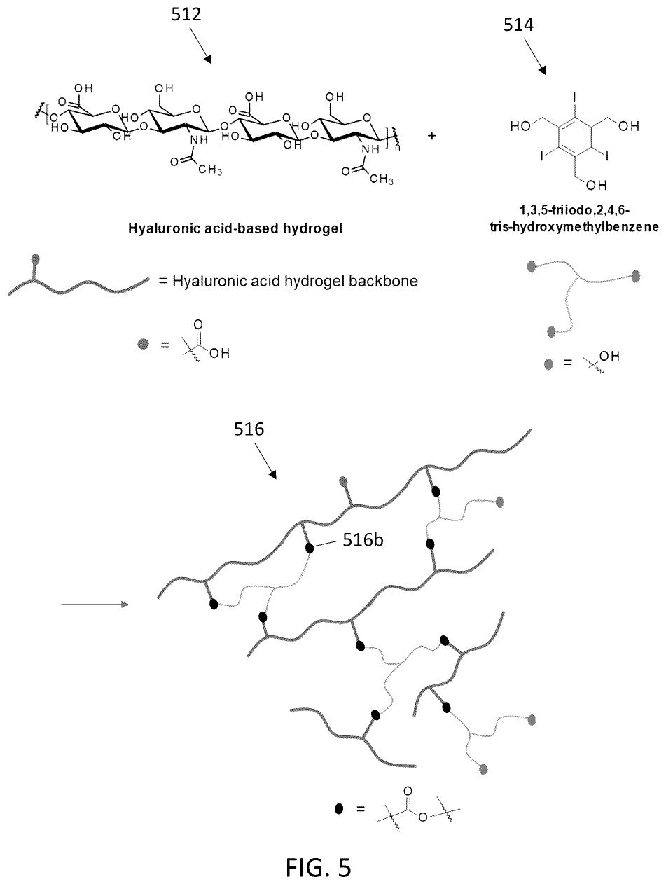

Further illustrative polyols for use in the present disclosure are described below. Such polyols may be used to form multi-arm polymeric polyols as described, for example, in FIGS. 3 and 4 above. Such polyols may also be used directly as crosslinkers as described, for example, in conjunction with FIG. 5 below.

Further polyols may be selected, for example, from sugars (monosaccharides, disaccharides, trisaccharides, etc.), sugar alcohols, calixarenes, polyhedral oligomeric silsesquioxanes (POSS), cyclodextrins, polyhydroxylated polymers, catechins, flavanols, anthocyanins, stilbenes, and polyphenols, among others.

Further polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Examples include C₁-C₈ alkane diols, including alpha, omega-C₂-C₈ alkane diols, such as 1,2-ethane diol (ethylene glycol), 1,3-propane diol, 1,4-butane diol, 1,5-pentane diol, 1,6-hexane diol, etc. triols such as methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, polyhedral oligomeric silsesquioxanes (POSS), catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Further polyols further include iodinated polyols, which may be desirable where radiopacity is desired. Iodinated polyols include iodinated aromatic polyols, examples of which are compounds that comprise 2 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

Specific examples of iodinated polyols for use in the present disclosure include iodinated polyols that are known for use as iodinated contrast agents, whose biocompatibility has been demonstrated to be reasonably well tolerated. Specific examples of iodinated polyols include commercially available 1,3,5-triiodo-2,4,6-trishydroxymethylbenzene (CAS #178814-33-0),

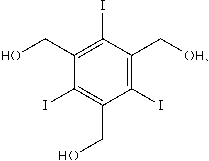 [SMILES: OCc1c(I)c(CO)c(I)c(CO)c1I]

iodixanol (CAS #92339-11-2),

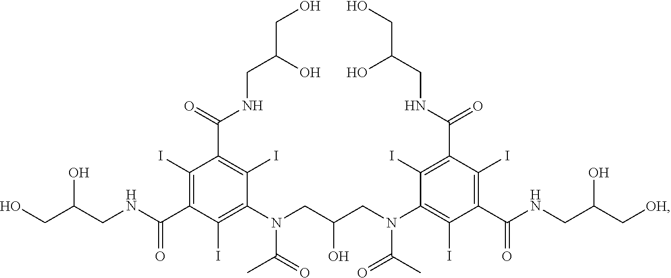 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

iotrolan (CAS #79770-24-4),

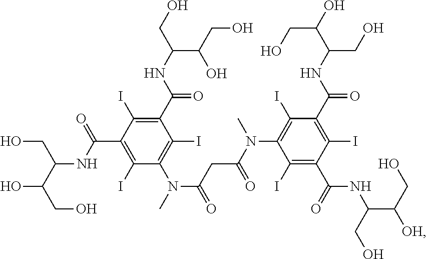 [SMILES: CN(C(=O)CC(=O)N(C)c1c(I)c(C(=O)NC(CO)C(O)CO)c(I)c(C(=O)NC(CO)C(O)CO)c1I)c1c(I)c(C(=O)NC(CO)C(O)CO)c(I)c(C(=O)NC(CO)C(O)CO)c1I]

iohexol (CAS #66108-95-0),

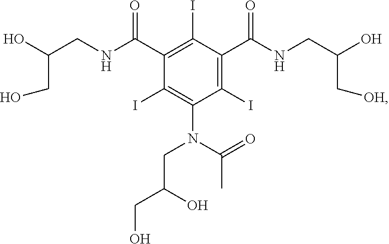 [SMILES: CC(=O)N(CC(O)CO)c1c(I)c(C(=O)NCC(O)CO)c(I)c(C(=O)NCC(O)CO)c1I]

ioversol (CAS #87771-40-2),

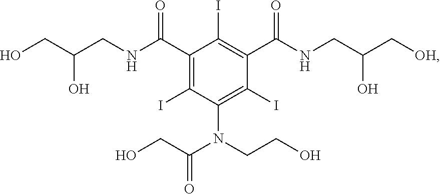 [SMILES: O=C(NCC(O)CO)c1c(I)c(C(=O)NCC(O)CO)c(I)c(N(CCO)C(=O)CO)c1I]

iopamidol (CAS #60166-93-0),

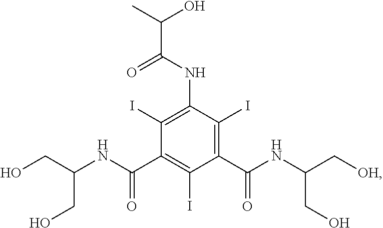 [SMILES: CC(O)C(=O)Nc1c(I)c(C(=O)NC(CO)CO)c(I)c(C(=O)NC(CO)CO)c1I]

iohexol impurity J (CAS #76801-93-9),

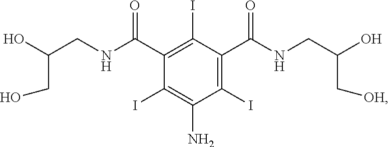 [SMILES: Nc1c(I)c(C(=O)NCC(O)CO)c(I)c(C(=O)NCC(O)CO)c1I]

and iopromide (CAS #73334-07-3),

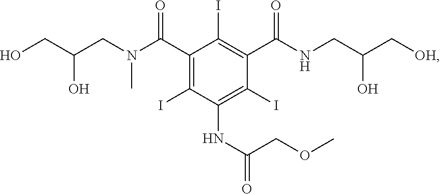 [SMILES: COCC(=O)Nc1c(I)c(C(=O)NCC(O)CO)c(I)c(C(=O)N(C)CC(O)CO)c1I]

among others.

As noted above, the preceding polyols may be used directly as crosslinkers for carboxylic-acid-containing polysaccharides. For example, referring now to FIG. 5, carboxyl groups of a carboxylic-acid-containing polysaccharide, specifically, hyaluronic acid (512) may be reacted with hydroxyl groups of a polyol, specifically 1,3,5-triiodo-2,4,6-trishydroxymethylbenzene (514), in an ester coupling reaction in the presence of a suitable coupling agent, such as DCC or EDC, to form a crosslinked polysaccharide (516) in which hyaluronic acid chains are crosslinked to one another through iodine-containing crosslinks that comprise hydrolysable ester bonds (516b).

Such a crosslinked polysaccharide hydrogel (516) may be formed in vivo within a subject or may be formed ex vivo and subsequently delivered to a subject. Such a crosslinked polysaccharide hydrogel (516) may also be contacted with a hydrolysis-accelerating catalytic composition that is adapted to break down ester bonds in the crosslinks as shown, for example, in FIG. 2B.

The preceding embodiments pertain to crosslinked polysaccharides in which a carboxylic-acid-containing polysaccharide is covalently bonded with a polyol through ester groups.

In other embodiments, a crosslinked polysaccharide may be formed by an ester-forming reaction between hydroxyl groups of a hydroxyl-containing polysaccharide and carboxyl groups of a polycarboxylic acid molecule. Because all polysaccharides contain hydroxyl groups, the range of polysaccharides that can be used in these embodiments extends to additional polysaccharides beyond carboxylic-acid-containing polysaccharides. Examples of such additional polysaccharides that can be crosslinked include cellulose derivatives including alkyl celluloses such as methyl cellulose and ethyl cellulose and hydroxyalkyl celluloses such as hydroxyethyl cellulose and hydroxypropyl cellulose, starches such as corn starch, potato starch, tapioca starch and cationic starch, chitosan and gelatin-polysaccharide composite materials, among others.

As above, the ester coupling between the carboxylic acid and hydroxyl groups may be performed in the presence of a suitable coupling agent, for example, a carbodiimide coupling agent, such as N,N′-dicyclohexylcarbodiimide (DCC), 1-Ethyl-3-(3-dimethylaminopropyl) carbodiimide (EDC), or N,N′-Diisopropylcarbodiimide (DIC), among others. The ester linkage that is formed is hydrolysable.

Polycarboxylic acid molecules for use in accordance with the present disclosure include polycarboxylic acid molecules having two or more carboxylic acid groups, for example, containing anywhere from 2 to 100 carboxylic acid groups (e.g., having 3 to 4 to 5 to 6 to 7 to 8 to 9 to 10 to 12 to 15 to 20 to 25 to 30 to 40 to 50 to 60 to 70 to 80 to 90 to 100 carboxylic acid groups.

Polycarboxylic acid molecules for use in the present disclosure include polymeric polycarboxylic acid molecules and non-polymeric polycarboxylic acid molecules.

Polycarboxylic acid molecules for use in the present disclosure include small molecule polycarboxylic acid molecules and large molecule polycarboxylic acid molecules.

Polymeric polycarboxylic acid molecules include linear polymers that comprise one or more polymer segments and have a carboxylic-acid-containing moiety linked at each end.

Polymeric polycarboxylic acid molecules also include multi-arm polymers that have a core region and a plurality of polymer arms, each polymer arm comprising one or more polymer segments and each arm having a fixed end linked to the core region and an opposite free end that is linked to a carboxylic-acid-containing moiety.

Polymer segments for use in the linear and multi-arm polymeric polycarboxylic acid molecules include those described above.

In certain embodiments, the core region comprises a residue of an iodinated or non-iodinated polyol comprising two or more hydroxyl groups, which may be used to form polymer arms as described above.

In some embodiments, polymeric polycarboxylic acid molecules can be formed from linear and multi-arm polymeric polyols as described above by reacting the terminal hydroxyl groups of the polymeric polyols with a cyclic anhydride (e.g., glutaric anhydride, succinic anhydride, malonic anhydride, adipic anhydride, diglycolic anhydride, 1,3-acetonedicarboxylic acid anhydride, etc.) to form carboxylic-acid-terminated linear and multi-arm polymers such as glutaric-acid-terminated linear and multi-arm polymers, succinic-acid-terminated linear and multi-arm polymers, malonic-acid-terminated linear and multi-arm polymers, adipic-acid-terminated linear and multi-arm polymers, diglycolic-acid-terminated linear and multi-arm polymers, 1,3-acetonedicarboxylic-acid-terminated linear and multi-arm polymers, and so forth. The preceding cyclic anhydrides, among others, may be reacted with a hydroxyl-terminated polymer under basic conditions to form a carboxylic-acid-terminated polymer comprising carboxylic acid end groups that are linked to the polymer arms through a hydrolysable ester bond (which is in addition to the hydrolysable ester bonds described elsewhere herein).

Further polycarboxylic acid molecules may be selected, for example, from non-iodinated polycarboxylic acid molecules having two or more carboxylic acid groups, including dicarboxylic acids such as C₁-C₈ alkane dicarboxylic acids, including alpha,omega-C₂-C₈ alkane dicarboxylic acids, such as 1,2-ethane dicarboxylic acid, 1,3-propane dicarboxylic acid, 1,4-butane dicarboxylic acid, 1,5-pentane dicarboxylic acid, 1,6-hexane dicarboxylic acid, etc., tricarboxylic acids such as propane-1,2,3-tricarboxylic acid, benzene-1,3,5-tricarboxylic acid, tetracarboxylic acids, pentacarboxylic acids, hexacarboxylic acids, heptacarboxylic acids, octacarboxylic acids, and so forth. Non-iodinated polycarboxylic acid molecules having two or more carboxylic acid groups can also be formed from any of the above-described non-iodinated polyols having two or more hydroxyl group by reacting the hydroxyl groups of the polyols with a cyclic anhydride to form carboxylic-acid groups.

Further polycarboxylic acid molecules include iodinated polycarboxylic acid molecules. Iodinated polycarboxylic acid molecules include iodinated aromatic polycarboxylic acid molecules, examples of which are compounds that comprise two or more carboxylic acid groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more carboxylic acid groups, which may be directly substituted to the aromatic groups or may be provided in the form of carboxyalkyl groups (e.g., C₁-C₄-carboxyalkyl groups containing one, two, three or four carbon atoms and containing one, two, three or four or more carboxylic acid groups). The carboxyalkyl groups may be linked to the aromatic group directly or through any suitable linking moiety, which may be selected, for example, from amide groups, amine groups, ether groups, ester groups, or carbonate groups, among others.

Iodinated polycarboxylic acid molecules having two or more carboxylic acid groups can be formed from any of the above-described iodinated polyols having two or more hydroxyl group by reacting the hydroxyl groups of the polyols with a cyclic anhydride to form carboxylic-acid groups.

Specific examples of iodinated polycarboxylic acid molecules for use in the present disclosure include 2,4,6-Triiodobenzene-1,3,5-tricarboxylic acid,

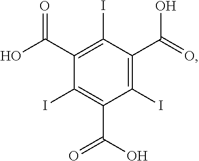 [SMILES: O=C(O)c1c(I)c(C(=O)O)c(I)c(C(=O)O)c1I]

2,4,6-Triiodo-1,3-benzenedicarboxylic acid (CAS #53451-54-0),

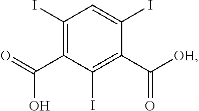 [SMILES: O=C(O)c1c(I)cc(I)c(C(=O)O)c1I]

and 2,4,5,6-Tetraiodo-1,3-benzenedicarboxylic acid (CAS #162321-54-2),

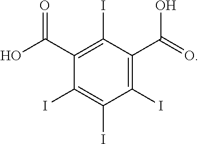 [SMILES: O=C(O)c1c(I)c(I)c(I)c(C(=O)O)c1I]

As previously indicated, the present disclosure pertains to compositions, devices, systems and methods for accelerating the hydrolysis (and thus the breakdown) of crosslinked polysaccharide hydrogels within a subject. In various embodiments, crosslinks between polysaccharide molecules in the crosslinked polysaccharide hydrogel contain hydrolysable ester bonds. The method comprises contacting the crosslinked polysaccharide hydrogel with a hydrolysis-accelerating catalytic composition that is adapted to accelerate hydrolysis of the ester bonds within the crosslinks.

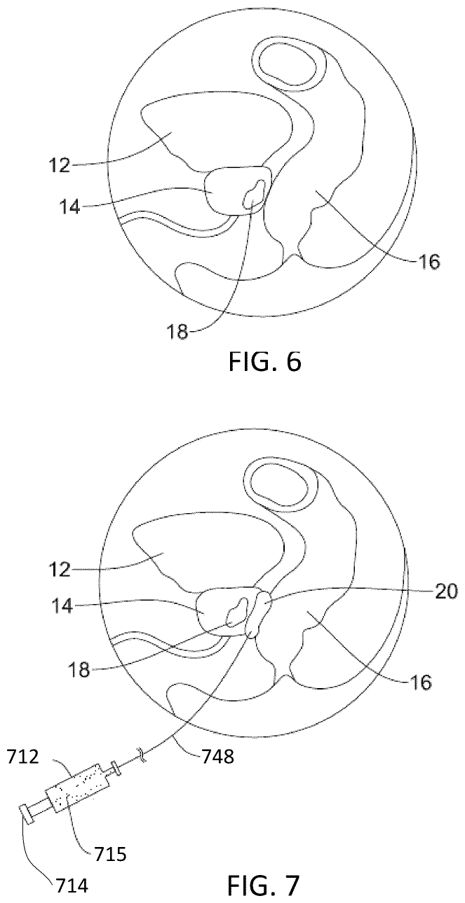

FIGS. 6 and 7 illustrate the placement of one such crosslinked polysaccharide hydrogel between the prostate and rectum of a subject. FIG. 6 is a schematic overview depicting a region of the human body including, for example, the bladder 12, prostate 14, and rectum 16. In this example, the prostate 14 may include a tumor 18. In some instances, it may be desirable to treat the tumor 18 with radiation therapy.

Prior to radiation therapy, it may be desirable to place a crosslinked polysaccharide hydrogel spacer 20 in the human body. As shown in FIG. 7, a preloaded syringe, in which an injectable crosslinked polysaccharide hydrogel composition 715 is disposed within a delivery syringe system that includes a syringe barrel 712 and a plunger 714, may be connected to a needle 748 the distal tip of which is positioned between the prostate 14 and rectum 16. The plunger 714 may be used to deliver the crosslinked polysaccharide hydrogel composition 715 to form a crosslinked polysaccharide hydrogel spacer 20. With the prostate 14 spaced from the rectum 16, radiation therapy may be used to treat the tumor 18.

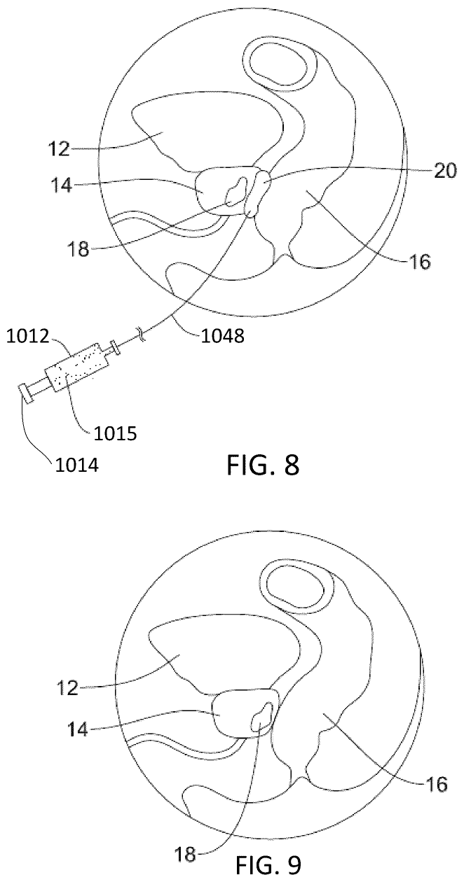

In the event that hydrolysis of the crosslinked polysaccharide hydrogel spacer 20 is desired, a hydrolysis-accelerating catalytic composition may be contacted with the crosslinked polysaccharide hydrogel spacer 20 in order to accelerate hydrolysis of the same. As shown in FIG. 8, a preloaded syringe, in which a hydrolysis-accelerating catalytic composition 1015 is disposed within a hydrolysis syringe system that includes a syringe barrel 1012 and a plunger 1014, may be connected to a needle 1048 the end of which is positioned in the crosslinked polysaccharide hydrogel spacer 20. The plunger 1014 may be used to deliver the hydrolysis-accelerating catalytic composition 1015 to the crosslinked polysaccharide hydrogel spacer 20 as schematically depicted in FIG. 8. The hydrolysis-accelerating catalytic composition 1015 may accelerate the hydrolysis of the crosslinked polysaccharide hydrogel spacer 20 as schematically depicted in FIG. 9.

It is noted that in some cases (e.g., wherein it is immediately apparent that the crosslinked polysaccharide hydrogel spacer 20 has been misplaced), the needle 1048 may be the same as the needle 748 that was used to initially introduce the crosslinked polysaccharide hydrogel spacer 20, allowing the preloaded syringe 710 to be disconnected from the needle 748 and the preloaded syringe 1010 to be connected to the needle 748 without withdrawing needle 748 from the subject.

In some aspects, the present disclosure pertains to a system that comprises (a) an injectable or implantable composition comprising a pre-formed crosslinked polysaccharide hydrogel that comprises crosslinks between polysaccharide molecules that contain hydrolysable ester bonds and (b) a hydrolysis-accelerating catalytic composition that is adapted to accelerate hydrolysis of the hydrolysable ester bonds.

Various hydrolysis-accelerating catalytic compositions are described above and include at least one hydrolysis-accelerating catalyst. The hydrolysis-accelerating catalytic compositions may be provided in a suitable reservoir such as a syringe, vial or ampule. Whether supplied in a syringe, vial, ampule or other reservoir, the hydrolysis-accelerating catalytic compositions may be provided, for example, in dry form (e.g., powder form) or in fluid form, such as in a solution form, or in a multi-phase fluid such as a suspension of particles of the at least one hydrolysis-accelerating catalyst or in an oil/water or water/oil emulsion form, wherein hydrolysis-accelerating catalyst is predominantly present in the oil phase or the aqueous phase. The hydrolysis-accelerating catalytic compositions may further include one or more additional agents, including therapeutic agents, imaging agents, colorants, tonicity adjusting agents, suspension agents, wetting agents, and pH adjusting agents as described below.

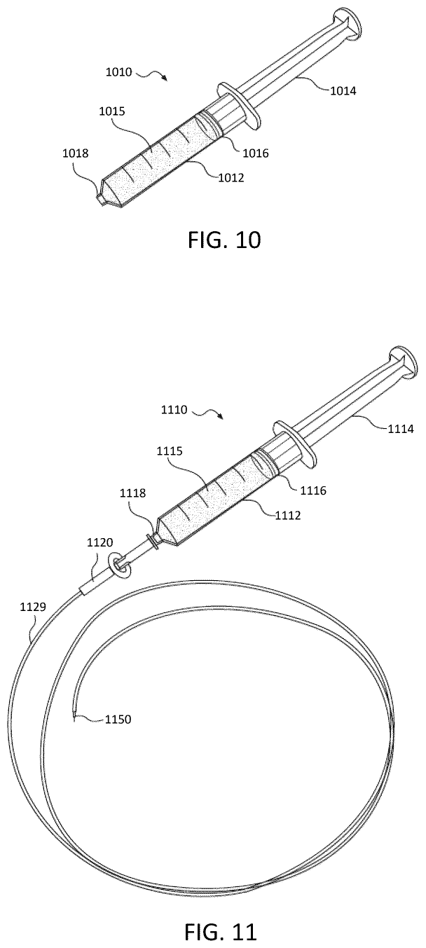

FIG. 10 illustrates a syringe 1010 for injection of a hydrolysis-accelerating catalytic composition as discussed above. The syringe 1010 may comprise a barrel 1012, a plunger 1014, and one or more stoppers 1016. The barrel 1012 may include a Luer adapter (or other suitable adapter/connector) at the distal end 1018 of the barrel 1012, suitable for attachment to an injection needle or a flexible catheter. The syringe barrel 1012 may serve as a reservoir, containing the hydrolysis-accelerating catalytic composition 1015 for injection into a subject, for example, through a needle or catheter.

Pre-formed crosslinked polysaccharide hydrogels can be formed by ester coupling between carboxylic acid groups of a carboxylic-acid-containing polysaccharide, such as one of those previously described, and hydroxyl groups of the polyol, such as one of those previously described. Pre-formed crosslinked polysaccharide hydrogels can also be formed by ester coupling between hydroxyl groups of a hydroxyl-containing polysaccharide, such as one of those previously described, and carboxyl groups of a polycarboxylic acid molecule, such as one of those previously described.

In various embodiments, such crosslinked polysaccharide hydrogels have a radiopacity that is greater than 100 Hounsfield units (HU), beneficially anywhere ranging from 100 HU to 500 HU to 750 HU to 1000 HU or more in various embodiments, for example, when measured on bench-top micro-CT systems such as Xtreme CT from Scanco Medical (Wangen-Brüttisellen, Switzerland) or similar.

Radiopacity can be provided, for example, by forming the crosslinked polysaccharide hydrogels from iodinated species such as those described above, or by including a separate radiocontrast agent.

Pre-formed crosslinked polysaccharides may be in any desired form, including a slab, a cylinder, a coating, or a particle. In some embodiments, the crosslinked polysaccharide is dried and then granulated into particles of suitable size. Granulating may be by any suitable process, for instance by homogenization, forcing through a screen, grinding (including cryogrinding), crushing, milling, pounding, or the like. Sieving or other known techniques can be used to classify and fractionate the particles. Crosslinked polysaccharide particles formed using the above and other techniques may varying widely in size, for example, having an average size ranging from 50 to 950 microns.

In addition to a crosslinked polysaccharide hydrogel as described above, preformed crosslinked polysaccharide hydrogel compositions in accordance with the present disclosure may contain additional agents, including therapeutic agents, imaging agents, colorants, tonicity adjusting agents, suspension agents, wetting agents, and pH adjusting agents as detailed below.

The pre-formed crosslinked polysaccharide hydrogel compositions may be provided in any suitable packaging. Where the crosslinked polysaccharide hydrogel composition is provided in injectable form (e.g., wherein the crosslinked polysaccharide hydrogel composition contains injectable crosslinked polysaccharide hydrogel particles, beads, pellets, etc.), the crosslinked polysaccharide hydrogel compositions may be provided in a reservoir, such as a syringe, vial or ampule. Whether supplied in a syringe, vial, ampule or other reservoir, the pre-formed crosslinked polysaccharide hydrogel compositions may be provided, for example, in dry form (e.g., in the form of a powder that contains crosslinked polysaccharide hydrogel particles) or in a fluid form (e.g., in the form of a suspension that contains crosslinked polysaccharide hydrogel particles).

The pre-formed crosslinked polysaccharide hydrogel compositions may be delivered to a subject using a suitable delivery device. Preferred subjects include mammalian subjects, particularly human subjects.

One exemplary delivery device is shown in FIG. 11, which illustrates a syringe 1110 providing a reservoir a pre-formed crosslinked polysaccharide hydrogel composition as discussed above. The syringe 1110 may comprise a barrel 1112, a plunger 1114, and one or more stoppers 1116. The barrel 1112 may include a Luer adapter (or other suitable adapter/connector), e.g., at the distal end 1118 of the barrel 1112, for attachment to an injection needle 1150 via a flexible catheter 1129. The proximal end of the catheter 1129 may include a suitable connection 1120 for receiving the barrel 1112. In other examples, the barrel 1112 may be directly coupled to the injection needle 1150. The syringe barrel 1112 may serve as a reservoir, containing a pre-formed crosslinked polysaccharide hydrogel composition 1115 for injection through the needle 1150.

In some embodiments, the crosslinked polysaccharide hydrogel compositions of the present disclosure can be imaged after administration using a suitable imaging technique such as ultrasound or an X-ray-based imaging technique, such as computerized tomography or X-ray fluoroscopy.

Because the crosslinked polysaccharide hydrogel compositions of the present disclosure have ester-containing crosslinks, degradation rate of the crosslinked polysaccharide hydrogel compositions will be tunable from slowly degraded over time from hydrolysis in aqueous bodily fluids to faster degradation based on the ester bonds density.

However, when desired, hydrolysis of the crosslinked polysaccharide hydrogel compositions of the present disclosure can be accelerated by contacting the crosslinked polysaccharide hydrogel compositions with a hydrolysis-accelerating catalytic composition as described herein.

The crosslinked polysaccharide hydrogel compositions described herein can be used for a number of medical purposes.

For example, the crosslinked polysaccharide hydrogel compositions can be injected to provide spacing between tissues, the crosslinked polysaccharide hydrogel compositions can be injected to provide fiducial markers, the crosslinked polysaccharide hydrogel compositions can be injected for tissue augmentation or regeneration, including cosmetic tissue augmentation, the crosslinked polysaccharide hydrogel compositions can be injected as a filler or replacement for soft tissue, the crosslinked polysaccharide hydrogel compositions can be injected to provide mechanical support for compromised tissue, the crosslinked polysaccharide hydrogel compositions can be injected as a scaffold, the crosslinked polysaccharide hydrogel compositions can be injected as lifting agents for internal cyst removal, and/or the crosslinked polysaccharide hydrogel compositions can be injected as a carrier of therapeutic agents in the treatment of diseases and cancers and the repair and regeneration of tissue, among other uses. The crosslinked polysaccharide hydrogel compositions can also be injected into a left atrial appendage during a left atrial appendage closure procedure. In some embodiments, crosslinked polysaccharide hydrogel compositions may be injected into the left atrial appendage after the introduction of a closure device such as the Watchman® left atrial appendage closure device available from Boston Scientific Corporation.

The crosslinked polysaccharide hydrogel compositions of the present disclosure may be used in a variety of medical procedures, including the following, among others: a procedure to implant a fiducial marker comprising a crosslinked polysaccharide hydrogel, a procedure to implant a tissue regeneration scaffold comprising a crosslinked polysaccharide hydrogel, a procedure to implant a tissue support comprising a crosslinked polysaccharide hydrogel, a procedure to implant a tissue bulking agent comprising a crosslinked polysaccharide hydrogel, a procedure to implant a therapeutic-agent-containing depot comprising a crosslinked polysaccharide hydrogel, a tissue augmentation procedure comprising implanting a crosslinked polysaccharide hydrogel, a procedure to introduce a crosslinked polysaccharide hydrogel between a first tissue and a second tissue to space the first tissue from the second tissue.

The crosslinked polysaccharide hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In various embodiments, kits are provided that include one or more delivery devices for delivering a pre-formed crosslinked polysaccharide hydrogel composition as described herein and a hydrolysis-accelerating catalytic composition as described herein to a subject. Such kits may include any of the following: one or more syringes, which may or may not contain the pre-formed crosslinked polysaccharide hydrogel composition or the hydrolysis-accelerating catalytic composition; one or more vials, which may or may not contain the pre-formed crosslinked polysaccharide hydrogel composition or the hydrolysis-accelerating catalytic composition; one or more needles (which may be compatible both with a delivery syringe system for delivering the pre-formed crosslinked polysaccharide hydrogel composition and a hydrolysis syringe system for delivering the hydrolysis-accelerating catalytic composition); one or more flexible tubes (which may be compatible both with a syringe for delivering the pre-formed crosslinked polysaccharide hydrogel composition and a syringe for delivering the hydrolysis-accelerating catalytic composition); and an injectable liquid such as water for injection, normal saline or phosphate buffered saline. Whether supplied in a syringe, vial, or other reservoir, the pre-formed crosslinked polysaccharide hydrogel composition and the hydrolysis-accelerating catalytic composition may independently be provided in dry form (e.g., powder form) or in a fluid form, which may be ready for injection.

In some aspects, the present disclosure pertains to a system that comprises (a) compositions for forming, within a subject, a crosslinked polysaccharide hydrogel that comprises polysaccharide molecules and crosslinks between polysaccharide molecules that contain hydrolysable ester bonds and (b) a hydrolysis-accelerating catalytic composition that is adapted to accelerate hydrolysis of the hydrolysable ester bonds.

In various embodiments, the crosslinked polysaccharide hydrogel that is formed within the subject has a radiopacity that is greater than 100 Hounsfield units (HU), beneficially anywhere ranging from 100 HU to 500 HU to 750 HU to 1000 HU or more in various embodiments, for example, when measured on bench-top micro CT systems such as Xtreme CT from Scanco Medical or similar.

As noted above, the hydrolysis-accelerating catalytic composition may be provided in a suitable reservoir such as a syringe, vial or ampule. Whether supplied in a syringe, vial, ampule or other reservoir, the hydrolysis-accelerating catalytic compositions may be provided, for example, in dry form (e.g., powder form) or in fluid form, such as in a solution form or in a multi-phase fluid such as a suspension of particles of the at least one hydrolysis-accelerating catalyst or in an oil/water or water/oil emulsion form, wherein hydrolysis-accelerating catalyst is predominantly present in the oil phase or the aqueous phase. The hydrolysis-accelerating catalytic composition may further include one or more additional agents, including therapeutic agents, imaging agents, colorants, tonicity adjusting agents, suspension agents, wetting agents, and pH adjusting agents as described below. A particular embodiment where a hydrolysis-accelerating catalytic composition 1015 is provided within a syringe 1010 is shown in FIG. 10 described above.

Compositions for forming a crosslinked polysaccharide hydrogel within a subject are described above and include a carboxylic-acid-containing polysaccharide as described above, a polyol as described above, and an ester coupling agent as described above.

The carboxylic-acid-containing polysaccharide, the polyol, or a combination of both may be provided in a suitable reservoir such as a syringe, vial or ampule. Whether supplied in a syringe, vial, ampule or other reservoir, the carboxylic-acid-containing polysaccharide, the polyol, or a combination of both may be provided, for example, in dry form (e.g., powder form) or in fluid form, such as in a solution form. Moreover, the carboxylic-acid-containing polysaccharide, the polyol, or a combination of both may further include one or more additional agents, including therapeutic agents, imaging agents, colorants, tonicity adjusting agents, suspension agents, wetting agents, and pH adjusting agents as described below.

The ester coupling agent also may be provided in a suitable reservoir such as a syringe, vial or ampule. Whether supplied in a syringe, vial, ampule or other reservoir, the ester coupling agent may be provided, for example, in dry form (e.g., powder form) or in fluid form, such as in a solution form. Moreover, the ester coupling agent may further include one or more additional agents, including therapeutic agents, imaging agents, colorants, tonicity adjusting agents, suspension agents, wetting agents, and pH adjusting agents as described below.

In some embodiments, the carboxylic-acid-containing polysaccharide, the polyol and the ester coupling agent are simultaneously administered to the subject, after which crosslinks are formed between the carboxylic-acid-containing polysaccharide, the polyol and the ester coupling agent.

In some embodiments, a system is provided which includes a delivery device that comprises a first reservoir that contains a first fluid composition that comprises the carboxylic-acid-containing polysaccharide and the polyol and a second reservoir that contains a second fluid composition that comprises the ester coupling agent. When the first and second fluid compositions are mixed, crosslinking commences between the carboxylic-acid-containing polysaccharide and the polyol. During operation, the first fluid composition and second fluid composition are dispensed from the first and second reservoirs and combined, whereupon the carboxylic-acid-containing polysaccharide and the polyol crosslink with one another to form a crosslinked polysaccharide hydrogel in the subject.

In various embodiments, a kit is provided that includes one or more delivery devices for delivering (a) a carboxylic-acid-containing polysaccharide as described herein, a polyol as described herein, and an ester coupling agent as described herein and (b) a hydrolysis-accelerating catalytic composition as described herein to a subject. Such kits may include any of the following: a syringe, vial or other container containing the carboxylic-acid-containing polysaccharide; a syringe, vial or other container containing the polyol; a syringe, vial or other container containing both the carboxylic-acid-containing polysaccharide and the polyol; a syringe, vial or other container containing the ester coupling agent; a syringe, vial or other container containing the hydrolysis-accelerating catalytic composition; one or more needles (which may be compatible with both a hydrolysis syringe system for delivering the hydrolysis-accelerating catalytic composition and a delivery syringe system for delivering the carboxylic-acid-containing polysaccharide, the polyol and the ester coupling agent); one or more flexible tubes (which may be compatible with both a hydrolysis syringe system for delivering the hydrolysis-accelerating catalytic composition and a delivery syringe system for delivering the carboxylic-acid-containing polysaccharide, the polyol and the ester coupling agent); an injectable liquid such as water for injection, normal saline or phosphate buffered saline which may be provided in a syringe, vial or other container.

Compositions for forming a crosslinked polysaccharide hydrogel within a subject further include a hydroxyl-containing polysaccharide as described above, a polycarboxylic acid molecule as described above, and an ester coupling agent as described above.

The hydroxyl-containing polysaccharide, the polycarboxylic acid molecule, or a combination of both may be provided in a suitable reservoir such as a syringe, vial or ampule. Whether supplied in a syringe, vial, ampule or other reservoir, the hydroxyl-containing polysaccharide, the polycarboxylic acid molecule, or a combination of both may be provided, for example, in dry form (e.g., powder form) or in fluid form, such as in a solution form. Moreover, the hydroxyl-containing polysaccharide, the polycarboxylic acid molecule, or a combination of both may further include one or more additional agents, including therapeutic agents, imaging agents, colorants, tonicity adjusting agents, suspension agents, wetting agents, and pH adjusting agents as described below.

As previously noted, the ester coupling agent also may be provided in a suitable reservoir such as a syringe, vial or ampule. Whether supplied in a syringe, vial, ampule or other reservoir, the ester coupling agent may be provided, for example, in dry form (e.g., powder form) or in fluid form, such as in a solution form. Moreover, the ester coupling agent may further include one or more additional agents, including therapeutic agents, imaging agents, colorants, tonicity adjusting agents, suspension agents, wetting agents, and pH adjusting agents as described below.

In some embodiments, the hydroxyl-containing polysaccharide, the polycarboxylic acid molecule and the ester coupling agent are simultaneously administered to the subject, after which crosslinks are formed between the hydroxyl-containing polysaccharide, the polycarboxylic acid molecule and the ester coupling agent.

In some embodiments, a system is provided which includes a delivery device that comprises a first reservoir that contains a first fluid composition that comprises the hydroxyl-containing polysaccharide and the polycarboxylic acid molecule and a second reservoir that contains a second fluid composition that comprises the ester coupling agent. When the first and second fluid compositions are mixed, crosslinking commences between the hydroxyl-containing polysaccharide and the polycarboxylic acid molecule. During operation, the first fluid composition and second fluid composition are dispensed from the first and second reservoirs and combined, whereupon the hydroxyl-containing polysaccharide and the polycarboxylic acid molecule crosslink with one another to form a crosslinked polysaccharide hydrogel in the subject.

In various embodiments, a kit is provided that includes one or more delivery devices for delivering (a) a hydroxyl-containing polysaccharide as described herein, a polycarboxylic acid molecule as described herein, and an ester coupling agent as described herein and (b) a hydrolysis-accelerating catalytic composition as described herein to a subject. Such kits may include any of the following: a syringe, vial or other container containing the hydroxyl-containing polysaccharide; a syringe, vial or other container containing the polycarboxylic acid molecule; a syringe, vial or other container containing both the hydroxyl-containing polysaccharide and the polycarboxylic acid molecule; a syringe, vial or other container containing the ester coupling agent; a syringe, vial or other container containing the hydrolysis-accelerating catalytic composition; one or more needles (which may be compatible with both a hydrolysis syringe system for delivering the hydrolysis-accelerating catalytic composition and a delivery syringe system for delivering the hydroxyl-containing polysaccharide, the polycarboxylic acid molecule and the ester coupling agent); one or more flexible tubes (which may be compatible with both a hydrolysis syringe system for delivering the hydrolysis-accelerating catalytic composition and a delivery syringe system for delivering the hydroxyl-containing polysaccharide, the polycarboxylic acid molecule and the ester coupling agent); an injectable liquid such as water for injection, normal saline or phosphate buffered saline which may be provided in a syringe, vial or other container.

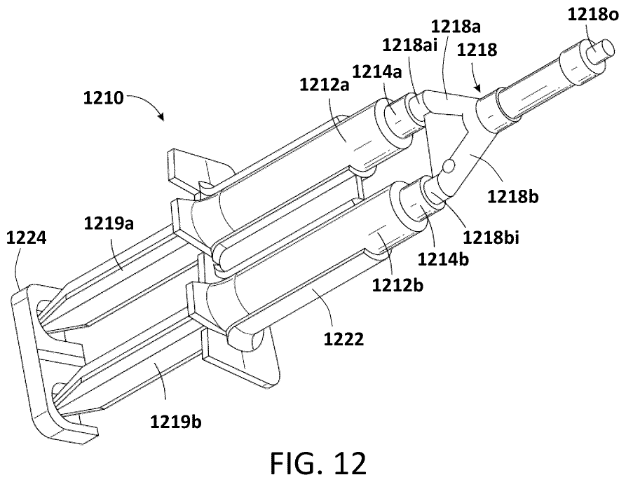

In particular embodiments, and with reference to FIG. 12, a syringe delivery system may include a delivery device 1210 that comprises a double-barrel syringe, which includes a first barrel 1212a having a first barrel outlet 1214a, which first barrel contains one of the first fluid compositions described above, a first plunger 1219a that is movable in the first barrel 1212a, a second barrel 1212b having a second barrel outlet 1214b, which second barrel 1212b contains one of the second fluid compositions described above, and a second plunger 1219b that is movable in the second barrel 1212b. In some embodiments, the device 1210 may further comprise a mixing section 1218 having a first mixing section inlet 1218ai in fluid communication with the first barrel outlet 1214a, a second mixing section inlet 1218bi in fluid communication with the second barrel outlet, and a mixing section outlet 12180. Also shown are a syringe holder 1222 configured to hold the first and second syringe barrels 1212a, 1212b, in a fixed relationship and a plunger cap 1224 configured to hold the first and second plungers 1219a, 1219b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject.

In either approach, a fluid admixture of the first and second fluid compositions is created which leads to the formation of a crosslinked polysaccharide hydrogel composition in the subject.

In some embodiments, the crosslinked polysaccharide hydrogel compositions of the present disclosure can be imaged after administration using a suitable imaging technique such as ultrasound or an X-ray-based imaging technique, such as computerized tomography or X-ray fluoroscopy.

Moreover, because the crosslinked polysaccharide hydrogel compositions of the present disclosure have ester-containing crosslinks, the crosslinked polysaccharide hydrogel compositions will be slowly degraded over time from hydrolysis in aqueous bodily fluids. However, when desired, hydrolysis of the crosslinked polysaccharide hydrogel compositions of the present disclosure can be accelerated by contacting the crosslinked polysaccharide hydrogel compositions with a hydrolysis-accelerating catalytic composition as described herein.

In either approach, first and second fluid compositions or a fluid admixture thereof is introduced into the patient for a variety of medical purposes.

For example, the first and second fluid compositions or a fluid admixture thereof can be injected to provide spacing between tissues, the first and second fluid compositions or a fluid admixture thereof can be injected to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injection for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked polysaccharide hydrogel is ultimately formed at the administration location.

After administration, the compositions of the present disclosure can be imaged using a suitable imaging technique such as ultrasound or an X-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked polysaccharide hydrogel composition, a procedure to implant a tissue regeneration scaffold comprising a crosslinked polysaccharide hydrogel composition, a procedure to implant a tissue support comprising a crosslinked polysaccharide hydrogel composition, a procedure to implant a tissue bulking agent comprising a crosslinked polysaccharide hydrogel composition, a procedure to implant an embolic composition comprising a crosslinked polysaccharide hydrogel composition, a procedure to implant a lifting agent comprising a crosslinked polysaccharide hydrogel composition, a procedure to introduce a left atrial appendage closure composition comprising a crosslinked polysaccharide hydrogel composition, a procedure to implant a therapeutic-agent-containing depot comprising a crosslinked polysaccharide hydrogel composition, a tissue augmentation procedure comprising implanting a crosslinked polysaccharide hydrogel composition, a procedure to introduce a crosslinked polysaccharide hydrogel composition between a first tissue and a second tissue to space the first tissue from the second tissue.

The first and second fluid compositions or a fluid admixture thereof may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

As noted above, additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly (DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

1. A kit for delivering and hydrolyzing a crosslinked polysaccharide hydrogel, the kit comprising (a) a hydrogel delivery system configured to deliver a crosslinked polysaccharide hydrogel to a subject, the crosslinked polysaccharide hydrogel comprising polysaccharide molecules that are crosslinked by crosslinks that comprise hydrolysable ester bonds and (b) a hydrogel hydrolysis system configured to deliver a hydrolysis-accelerating catalytic composition to the subject, the hydrolysis-accelerating catalytic composition adapted to accelerate hydrolysis of the hydrolysable ester bonds in the crosslinked polysaccharide hydrogel.

2. The kit of claim 1, wherein the hydrolysis-accelerating catalytic composition comprises a hydrolysis-accelerating catalyst selected from a hydroxide catalyst and an enzymatic protein.

3. The kit of claim 1, wherein the hydrolysis-accelerating catalytic composition is prepackaged in a hydrolysis syringe system.

4. The kit of claim 1, wherein the hydrogel hydrolysis system further comprises a needle, a flexible tube, or both and wherein the hydrolysis syringe system is configured for coupling to the needle, the flexible tube, or both.

5. The kit of claim 1, wherein the hydrogel delivery system comprises a preformed crosslinked polysaccharide hydrogel.

6. The kit of claim 5, wherein the preformed crosslinked polysaccharide hydrogel comprises crosslinked polysaccharide hydrogel particles.

7. The kit of claim 6, wherein the preformed crosslinked polysaccharide hydrogel particles are prepackaged in a delivery syringe system.

8. The kit of claim 1, wherein the hydrogel delivery system comprises a carboxylic-acid-containing polysaccharide, a polyol, and an ester coupling agent.

9. The kit of claim 8, wherein the hydrogel delivery system is configured to form a mixture of the carboxylic-acid-containing polysaccharide, the polyol, and the ester coupling agent and to deliver the mixture to a subject whereupon the carboxylic-acid-containing polysaccharide and the polyol form said crosslinks with one another.

10. The kit of claim 8, wherein the hydrogel delivery system comprises a delivery syringe system that is configured to form a mixture of the carboxylic-acid-containing polysaccharide, the polyol, and the ester coupling agent and to deliver the mixture to a subject whereupon the carboxylic-acid-containing polysaccharide and the polyol form said crosslinks with one another.

11. The kit of claim 10, wherein the delivery syringe system comprises a dual barrel syringe device for creating and delivering the mixture of the carboxylic-acid-containing polysaccharide, the polyol, and the ester coupling agent.

12. The kit of claim 1, wherein the hydrogel delivery system comprises a hydroxyl-containing polysaccharide, a polycarboxylic acid molecule, and an ester coupling agent.

13. The kit of claim 12, wherein the hydrogel delivery system is configured to form a mixture of the hydroxyl-containing polysaccharide, the polycarboxylic acid molecule, and the ester coupling agent and to deliver the mixture to a subject whereupon the hydroxyl-containing polysaccharide and the polycarboxylic acid molecule form said crosslinks with one another.

14. The kit of claim 12, wherein the hydrogel delivery system comprises a delivery syringe system that is configured to form a mixture of the hydroxyl-containing polysaccharide, the polycarboxylic acid molecule, and the ester coupling agent and to deliver the mixture to a subject whereupon the hydroxyl-containing polysaccharide and the polycarboxylic acid molecule form said crosslinks with one another.

15. The kit of claim 14, wherein the delivery syringe system comprises a dual barrel syringe device for creating and delivering the mixture of the hydroxyl-containing polysaccharide, the polycarboxylic acid molecule, and the ester coupling agent.

16. A method comprising (a) delivering a crosslinked polysaccharide hydrogel to a subject, the crosslinked polysaccharide hydrogel comprising polysaccharide molecules that are crosslinked by crosslinks that comprise hydrolysable ester bonds and (b) delivering a hydrolysis-accelerating catalytic composition to the subject such that the hydrolysis-accelerating catalytic composition contacts the crosslinked polysaccharide hydrogel, the hydrolysis-accelerating catalytic composition acting to accelerate hydrolysis of the hydrolysable ester bonds in the crosslinked polysaccharide hydrogel.

17. The method of claim 16, comprising delivering the crosslinked polysaccharide hydrogel to the subject via a delivery syringe system coupled to a needle; disconnecting the delivery syringe system from the needle; attaching a hydrolysis syringe system to the needle, the hydrolysis syringe system including the hydrolysis-accelerating catalytic composition disposed in a syringe barrel; and injecting the hydrolysis-accelerating catalytic composition into the subject.

18. The method of claim 16, further comprising observing a complication associated with the delivering of the crosslinked polysaccharide hydrogel to the subject prior to injecting the hydrogel hydrolysis material into the subject.

19. The method of claim 18, wherein the complication includes misplacement of the crosslinked polysaccharide hydrogel and/or wherein the complication includes subject discomfort.

20. The method of claim 16, further comprising delivering a replacement crosslinked polysaccharide hydrogel into the subject.
